(19) United States
(12) Patent Application Publication
(10) Pub. No.: US 2026/0131101 A1
(43) Pub. Date: May 14, 2026

(54) RESPIRATORY GAS HUMIDIFIER FOR A VENTILATOR

(71) Applicant: Loewenstein Medical Technology S.A., Luxembourg (LU)

(72) Inventors: Joachim GARDEIN, Icod de los Vinos (ES); Stefan HEIN, Hamburg (DE)

(21) Appl. No.: 19/383,840

(22) Filed: Nov. 10, 2025

(30) Foreign Application Priority Data
Nov. 14, 2024 (DE) 10 2024 133 290.5

(51) Int. Cl. A61M 16/10; A61M 16/16

(52) U.S. Cl. CPC A61M 16/1075; A61M 16/16

(57) ABSTRACT

The invention relates to a respiratory gas humidifier having a housing comprising an upper housing part and a lower housing part with a water chamber for humidifying and/or warming up a respiratory gas. The humidifier further comprises a respiratory gas inlet and a respiratory gas outlet, a respiratory gas path for conducting the respiratory gas from the respiratory gas inlet to the respiratory gas outlet being formed between the respiratory gas inlet and outlet. The gas path is guided through the water chamber and comprises a watertight channel for conducting the humidified and/or warmed-up respiratory gas, said channel leading into the water chamber at one end and into the respiratory gas outlet at another end, and being configured such that the respiratory gas outlet is arranged on the lower housing part and/or formed at least partially thereby.

### CROSS-REFERENCE TO RELATED APPLICATIONS

The present application claims priority under 35 U.S.C. § 119 of German Patent Application No. 10 2024 133 290.5, filed Nov. 14, 2024, the entire disclosure of which is expressly incorporated by reference herein.

### BACKGROUND OF THE INVENTION

### 1. Field of the Invention

The invention relates to a respiratory gas humidifier for humidifying and/or warming up a respiratory gas. Furthermore, the invention relates to a ventilator having such a respiratory gas humidifier.

### 2. Discussion of Background Information

A respiratory gas humidifier is an apparatus for humidifying and/or warming up a respiratory gas. Patients who receive artificial respiration or respiratory support may perceive respiration with air that is too cold or too dry as uncomfortable. In addition, artificial respiration may result in dry airways. Therefore, it is advantageous when the respiratory gas to be inhaled is humidified and warmed up with the aid of a respiratory gas humidifier. In this way, it is possible to reduce or avoid drying out of the mucous membranes and the acceptance of respiration or respiratory support by the patent may be increased.

Respiratory gas humidifiers have a liquid store in order for it to be possible to humidify the respiratory gas over a relatively long period of time. In order to prevent undesired or uncontrolled escape of liquid from the device, the device should exhibit a certain stability and, moreover, be designed such that the liquid cannot escape from the device in an uncontrolled manner even when the device is inclined.

In view of the foregoing, it would be advantageous to have available an improved respiratory gas humidifier for humidifying and/or warming up a respiratory gas.

### SUMMARY OF THE INVENTION

In a first aspect, the invention provides a respiratory gas humidifier for humidifying and/or warming up a respiratory gas, in particular for use with a ventilator. The respiratory gas humidifier comprises a housing having an upper housing part and a lower housing part with a water chamber for humidifying and/or warming up a respiratory gas. The respiratory gas humidifier comprises a respiratory gas inlet and a respiratory gas outlet, wherein a respiratory gas path for conducting the respiratory gas from the respiratory gas inlet to the respiratory gas outlet is formed between the respiratory gas inlet and the respiratory gas outlet, said respiratory gas path being guided through the water chamber. The respiratory gas path comprises a watertight channel for conducting the humidified and/or warmed-up respiratory gas, said channel leading into the water chamber at one end and into the respiratory gas outlet at another end. The channel is configured such that the respiratory gas outlet is arranged on the lower housing part and/or formed at least partially thereby.

A “respiratory gas” can be a pure respiratory gas or a respiratory gas mixture made up of a plurality of respiratory gases, with which a patient can receive respiration. The respiratory gas may be or comprise normal ambient air, oxygen, nitrogen, carbon dioxide, anaesthetic gas, steam, or a combination of at least two of these components.

“Respiratory gas humidification” can be understood as meaning that the respiratory gas is humidified by controlled addition of water or an aqueous solution, preferably in combination with warming up of the respiratory gas. This can be realized in that the respiratory gas is conducted over a liquid surface such that the liquid releases water or steam to the respiratory gas.

The term “water chamber” is intended herein to denote generally a container for storing a liquid (water or an aqueous liquid). The water chamber may comprise an electrically controllable heating element, for example a heating rod or a heating plate, for heating the liquid, such that the liquid can be evaporated in a targeted manner and picked up by the respiratory gas. It is also possible for a flow conducting element to be arranged in the water chamber, said flow conducting element being designed to conduct the respiratory gas in the direction of the liquid surface. The water chamber may comprise filling level indicators which indicate a maximum and/or minimum filling height for the liquid. The water chamber may have a water chamber inlet and a water chamber outlet. The respiratory gas can be guided into the water chamber through the water chamber inlet and out of the water chamber through the water chamber outlet.

A “respiratory gas path” should be understood as meaning those regions of the respiratory gas humidifier that are designed to receive and/or conduct the respiratory gas. The respiratory gas path may be formed, at least for the most part, within the housing of the respiratory gas humidifier. The respiratory gas path may be formed, for example, by channels, chambers, passages/passageways, openings and the like, which are connected pneumatically together. The respiratory gas path is—apart from the inlet and the outlet—formed in a respiratory-gastight manner with respect to its surroundings.

The respiratory gas inlet may be or comprise the start of the respiratory gas path. The respiratory gas inlet may be configured to introduce the respiratory gas into the respiratory gas humidifier. The respiratory gas outlet may be or comprise the end of the respiratory gas path. The respiratory gas outlet may be configured to conduct the respiratory gas out of the respiratory gas humidifier.

Via the respiratory gas inlet and/or the respiratory gas outlet, the respiratory gas humidifier can be connected to further channels, hoses and/or to components of a ventilator in an airtight manner. The respiratory gas inlet and respiratory gas outlet can thus serve as ports via which the respiratory gas humidifier can be connected to further components in an airtight manner.

From the respiratory gas outlet, the warmed-up and/or humidified respiratory gas can be guided directly to the patient. The respiratory gas outlet can also be connected to a ventilator, such that the warmed-up and/or humidified respiratory gas can be guided to the patient via the ventilator. The connection to the patient can also be realized via one or more breathing hoses and/or a suitable patient interface, which can be connected to the respiratory gas outlet and/or to the ventilator.

Within the meaning of the invention, a “patient interface” can be any peripheral device which is designed to interact with a living being. The patient interface may be, for example, a respiratory mask such as a nasal mask, nasal pillow mask, nasal cannula or oxygen cannula, full-face or total-face mask, or a tracheal tube or a tracheal cannula.

A ventilator can be any device that supports the natural breathing of a living being and/or carry out respiration and/or is used for respiratory therapy and/or is used for inhalation anaesthesia and/or acts in some other way on the breathing of the patient. A ventilator may be, for example, a ventilator for clinical or domestic applications, a respiratory therapy device, a CPAP, APAP or BiLevel device, a high-flow therapy device, an anaesthetic device, an emergency ventilator, an oxygen-supplying device, a diagnostic system or a cough therapy device or cough-assist machine.

Since the respiratory gas outlet is arranged on the lower housing part of the respiratory gas humidifier and/or formed thereby, it is also possible for components connected to the respiratory gas outlet of the respiratory gas humidifier to be attached to the lower housing part. This affords the advantage that it is not possible, or possible only with difficulty, for the components connected to the respiratory gas outlet to destabilize the respiratory gas humidifier in the event of a pulling movement. The risk of the respiratory gas humidifier tipping or falling over if the lower part of the device is pulled on is minimized. This thus generally affords the advantage of good stability of the respiratory gas humidifier.

According to one embodiment, the respiratory gas outlet—when the water chamber has been filled as intended with a liquid for humidifying the respiratory gas—may be arranged below the liquid surface.

According to one embodiment, the channel may be formed at least partially by the upper housing part and/or the lower housing part or extend through same.

A “channel” may be an elongate tube, a hose or a combination of the two. The channel may have a rigid or flexible or partially flexible channel wall and be configured to conduct the respiratory gas. The channel is in the form of an elongate cavity with a wall enclosing a lumen of the channel. This wall may be formed by the upper housing part and/or the lower housing part.

The upper housing part and the lower housing part may be formed in particular in two parts. In order to put the respiratory gas humidifier into an operational state, the upper housing part and the lower housing part may be connected together in a respiratory-gastight manner. The connection may preferably be designed to be reversible, such that the respiratory gas humidifier can be taken apart for cleaning and/or filling. The connection may be realized via a reversibly designed connecting mechanism. The respiratory gas humidifier may also have a locking mechanism with which the connection of the individual housing parts can be locked in a respiratory-gastight manner.

According to one embodiment, the respiratory gas humidifier may have a central part which is arranged in a respiratory-gastight manner between the upper housing part and the lower housing part, wherein the channel may be formed at least partially by the central part or extend through same. According to one embodiment, the channel may be formed at least partially only by the connection of the upper housing part to the lower housing part or by the connection of the upper housing part to the lower housing part and to the central part.

The channel or the channel wall may be formed at least partially in one part or in a multipart manner, for example in two parts. This may make it easier to produce the channel and also to clean same. Upon reversal of the connection of the upper housing part, lower housing part and/or central part, the channel can thus be at least locally opened, in particular in order to clean it.

According to one embodiment, the channel may also comprise a heating device, which may be configured to heat the channel to a first temperature in order to avoid condensation and/or to a second temperature in order to kill off and/or inactivate germs. The channel may thus comprises, for example, a heating wire which extends along the channel and can output heat into the interior of the channel.

According to one embodiment, the channel may, as seen in its longitudinal direction, be formed in a straight manner and/or at least partially in an angled and/or bent manner. According to one embodiment, the channel may, as seen in its longitudinal direction, comprise one or more channel portions that are fluidically connected together, wherein the channel may comprise a first channel portion for connecting to the water chamber outlet, a second channel portion for connecting to the respiratory gas outlet, and at least one further channel portion connecting the first channel portion to the second channel portion, wherein the channel portions may be configured to effect at least one deflection of the respiratory gas.

Deflection of the respiratory gas may suppress noise. The number of channel portions may be chosen to be variable. In this case, the more deflections the respiratory gas undergoes, the quieter the respiratory gas humidifier can be designed to be.

According to one embodiment, the channel may be configured such that—when the respiratory gas humidifier is standing as intended on a bottom of the lower housing part—the first channel portion is arranged at least largely vertically and/or the second channel portion is arranged at least largely horizontally and/or a third channel portion is arranged at least largely horizontally and/or a fourth channel portion is arranged at least largely vertically. According to one embodiment, the first channel portion may be configured such that—when the respiratory gas humidifier is standing as intended on the bottom and the water chamber has been filled with a liquid for humidifying the respiratory gas—it leads away from the liquid surface in a vertical direction.

The channel is preferably configured such that no excess liquid can be entrained by the respiratory gas stream. The channel may also be configured such that, if the respiratory gas humidifier tips, the liquid cannot flow out of the water chamber into the channel—or can only do so with difficulty. For this purpose, the first channel portion may be arranged vertically. The first channel portion may be configured such that—when the respiratory gas humidifier is standing as intended on a bottom of the lower housing part—it is arranged orthogonally to the liquid surface.

According to one embodiment, the respiratory gas humidifier may be configured such that the respiratory gas inlet—when the respiratory gas humidifier is standing as intended on a bottom of the lower housing part—is arranged above the respiratory gas outlet as seen in the vertical direction. According to one embodiment, the respiratory gas humidifier may be configured such that the third channel portion—when the respiratory gas humidifier is standing as intended on a bottom of the lower housing part—is arranged above the respiratory gas outlet and/or above the second channel portion as seen in the vertical direction. Such an arrangement affords additional protection against uncontrolled ingress of water into the channel and/or into the respiratory gas outlet.

According to one embodiment, the channel may, as seen in its transverse direction, be formed in a straight manner and/or at least partially in an angled and/or bent manner. Thus, the respiratory gas path can lead from the middle of the respiratory gas humidifier to the housing and/or to the respiratory gas outlet. As a result of the channel being angled or bent in the transverse direction, the respiratory gas humidifier can be formed in a particularly space-saving manner. The channel may be configured to convey the respiratory gas from the water chamber outlet to the respiratory gas outlet during operation of the respiratory gas humidifier. A bend of the channel as seen in its transverse direction may be particularly advantageous since turbulence in the air circulation can be avoided and an airflow that is laminar (as far as possible) can arise in the channel.

According to one embodiment, the channel may at least partially have a separating element which is configured to subdivide the channel, as seen in its longitudinal direction, at least partially into at least two sub-channels. The separating element may be configured to calm the flow of respiratory gas in the channel. The separating element may be configured to promote a laminar flow in the channel.

According to one embodiment, the respiratory gas humidifier may comprise a water chamber inlet which may be formed in the upper housing part or in the central part, wherein the water chamber inlet may be in the form of a simple opening or of a water chamber inlet channel. In some embodiments, the water chamber inlet may be in the form of an opening in the upper housing part. According to one embodiment, the water chamber inlet may correspond to the respiratory gas inlet. In alternative embodiments, the water chamber inlet may also be in the form of an opening in the central part. Then, the respiratory gas inlet may be arranged in the upper housing part or be formed thereby. In such an embodiment, the respiratory gas can initially be introduced into the upper housing part via the respiratory gas inlet and from there into the water chamber via the water chamber inlet of the central part. In some embodiments, the water chamber inlet may have a volume and be in the form of a water chamber inlet channel in the upper housing part and/or in the central part.

According to one embodiment, the respiratory gas humidifier may comprise a chamber which may be formed between the respiratory gas inlet and the water chamber inlet and be part of the respiratory gas path, wherein a cross section of the chamber may be larger than a cross section of the respiratory gas inlet and/or a cross section of the water chamber inlet or of the water chamber inlet channel and/or of the channel. According to one embodiment, the chamber and/or the channel and/or the water chamber and/or the water chamber inlet or the water chamber inlet channel may have acoustic impedances and have different dimensions such that their respective acoustic impedances differ from one another.

The abovementioned different portions of the respiratory gas path may each differ from immediately adjoining portions of the respiratory gas path, in particular with regard to a size of their cross sections orthogonal to the direction of flow. It is preferred here for a cross section and/or a shape of the respiratory gas path to abruptly decrease or increase at a transition between two adjoining portions of the respiratory gas path.

Sound can be absorbed by the resultant different acoustic impedances. An “acoustic impedance”, in particular an acoustic flow impedance, denotes the resistance that is set against sound propagation. Reflections of the sound waves occur at the transition between regions with different acoustic impedances, resulting in a sound-absorbing effect.

According to one embodiment, the water chamber outlet and/or the first channel portion may be arranged, as seen in a horizontal plane, in the region of the middle of the respiratory gas humidifier. The respiratory gas humidifier is designed such that the water chamber outlet and/or the first channel portion is arranged at a distance from the liquid surface of the water chamber. The maximum fill height is chosen such that the liquid does not reach the water chamber outlet. In addition, the water chamber outlet and/or the first channel portion is/are arranged centrally in the device, such that the liquid cannot penetrate into the channel in an uncontrolled manner during operation and/or transport.

The respiratory gas humidifier is in the form of a three-dimensional body and may comprise a bottom and at least one side wall. The respiratory gas humidifier may, for example, have the basic shape of a cuboid, cube, prism, cylinder, pyramid or the like. The water chamber outlet may be arranged such that, in a horizontal plane, it is as far away as possible from the side wall or from all the side walls of the respiratory gas humidifier. The water chamber outlet may, as seen in a horizontal plane, be arranged at the centre of the respiratory gas humidifier. The respiratory gas humidifier can thus be designed such that it can be inclined as far as a certain angle without liquid being able to pass into the water chamber outlet and/or the channel. As a result of the water chamber outlet being arranged in the middle of the respiratory gas humidifier, it can be designed such that an inclination or tipping of the humidifier to all sides is tolerable without liquid being able to pass into the upper region of the respiratory gas humidifier.

The invention also provides a ventilator having a respiratory gas duct, a first port and a second port, comprising a respiratory gas humidifier according to the invention, wherein the respiratory gas humidifier is connected to the respiratory gas duct of the ventilator via a respiratory gas inlet at the first port and via a respiratory gas outlet at the second port.

A “ventilator” may be understood as being a device for respiration and/or for anaesthesia. The ventilator can thus exhibit a respiration function and an anaesthesia function and a combination thereof. “Respiration” may be understood as being artificial respiration, breathing support, respiratory therapy, respiratory diagnosis, cough assistance, an oxygen-supplying therapy, for example a high-flow therapy, inhalation anaesthesia and a combination of at least two of these examples. Thus, using the ventilator, an individual can receive respiration or respiratory support and alternatively or additionally also be kept anaesthetized. For this purpose, the ventilator can also be operated with anaesthetic gases and thus be used for inhalation anaesthesia.

A ventilator can thus be any device that supports the natural breathing of an individual and/or carries out respiration and/or is used for respiratory therapy and/or is used for inhalation anaesthesia and/or acts in some other way on the breathing of the patient or user. A ventilator may be designed for clinical or domestic applications and be, for example, a respiratory therapy device, a CPAP, APAP or BiLevel device, a high-flow therapy device, an anaesthetic device, an emergency ventilator, an oxygen-supplying device, a diagnostic system or a cough therapy device or cough-assist machine.

Within the meaning of the invention, a “patient interface” can be any peripheral device which is designed to interact with a living being. In particular, the patient interface is configured for respiration, therapeutic and/or diagnostic purposes in conjunction with the ventilator. The patient interface may be in the form of a respiratory mask. These include, for example, but not exclusively, nasal masks, nasal pillow masks, nasal cannulas or oxygen cannulas, full-face or total-face masks and tracheal tubes or cannulas.

### BRIEF DESCRIPTION OF THE DRAWINGS

Embodiments of the invention are described in the following text with reference to the accompanying figures. Neither the description nor the figures should be understood as limiting the scope of the invention.

FIG. 1 shows a schematic depiction of a respiratory gas humidifier in cross section according to one embodiment of the invention.

FIG. 2 shows a perspective exterior view of a respiratory gas humidifier according to one embodiment of the invention.

FIG. 3 shows a perspective cross section of a respiratory gas humidifier according to one embodiment of the invention.

FIG. 4 shows a perspective exterior view of a respiratory gas humidifier without the upper housing part being illustrated, according to one embodiment of the invention.

FIG. 5 shows a perspective exterior view of a central part of a respiratory gas humidifier according to one embodiment of the invention.

FIG. 6 shows a view of a central part from above according to one embodiment of the invention.

FIG. 7 shows a view of a lower housing part from above according to one embodiment of the invention.

The drawings are purely schematic and sometimes not to scale. Where identical reference signs are used in different drawings, these reference signs denote identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

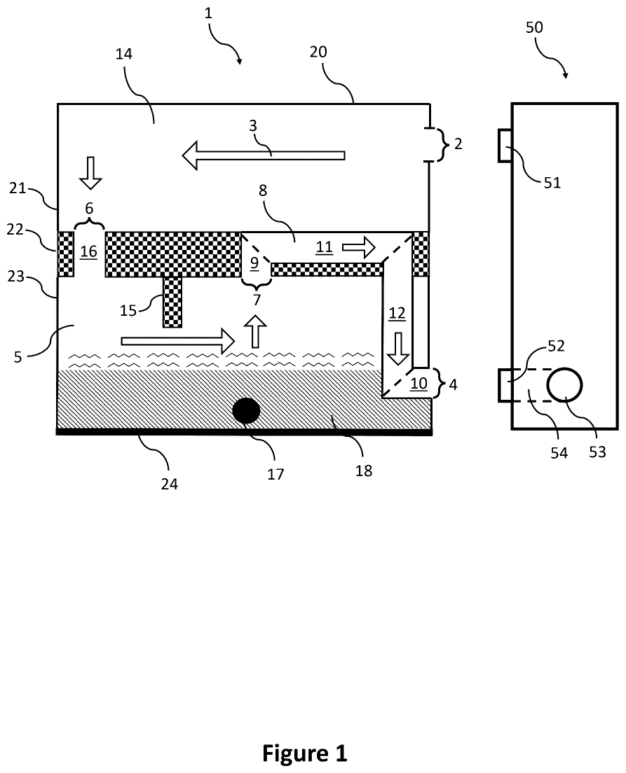

FIG. 1 shows a schematic depiction of a respiratory gas humidifier 1 in cross section according to one embodiment of the invention. The respiratory gas humidifier 1 is configured for use in combination with a ventilator 50. The respiratory gas humidifier 1 is configured to humidify and/or warm up a respiratory gas. In this example, the respiratory gas humidifier 1 has a two-part housing 20 consisting of a lower housing part 23 and an upper housing part 21. The lower housing part 23 comprises a bottom 24, which is configured for stably positioning the respiratory gas humidifier 1. As indicated, the bottom 24 can be formed in a planar manner. The bottom 24 may, for example, also be in the form of one or more feet with which the respiratory gas humidifier 1 can be positioned. The bottom 24 defines an underside of the respiratory gas humidifier 1. When in use as intended, the respiratory gas humidifier 1 stands on the bottom 24 of the lower housing part 23.

Furthermore, the respiratory gas humidifier 1 comprises a respiratory gas inlet 2 and a respiratory gas outlet 4. The respiratory gas inlet 2, when in use as intended, can be arranged above the respiratory gas outlet 4 as seen in the vertical direction. Formed between the respiratory gas inlet 2 and the respiratory gas outlet 4 is a respiratory gas path 3 for conducting the respiratory gas. The respiratory gas path 3 is indicated in FIG. 1 by way of block arrows. The arrows indicate the direction of flow of the respiratory gas. The respiratory gas humidifier 1 is configured to guide the respiratory gas along the respiratory gas path 3.

The respiratory gas inlet 2 may be configured to be connected to a device having a respiratory gas drive. Such a device is referred to as a ventilator 50 in the following text. The ventilator 50 may comprise a first port 51 for connecting to the respiratory gas inlet 2. Thus, the respiratory gas can flow out of the ventilator 50 and into the interior of the respiratory gas humidifier 1 via the respiratory gas inlet 2. The respiratory gas can thus enter the respiratory gas path 3. Via the respiratory gas outlet 4, the humidified and/or warmed-up respiratory gas can flow out of the interior of the respiratory gas humidifier 1. The respiratory gas can thus exit the respiratory gas path 3 and be used for the respiration of a patient.

The respiratory gas outlet 4 can likewise be configured to be connected to the ventilator 50. Then, the respiratory gas can flow out of the interior of the respiratory gas humidifier 1 back into the ventilator 50 and from there be used for the respiration of the patient. For this purpose, the ventilator 50 may comprise a second port 52 for connecting to the respiratory gas outlet 4. In addition, the ventilator 50 may have a hose port 53 and a line 54, which is formed between the second port 52 and the hose port 53. Before the respiratory gas is conducted to the patient, the pressure and/or the respiratory gas flow and/or the volume can usually be measured. For this purpose, the respiratory gas humidifier 1 and/or the ventilator 50 may have corresponding sensors.

In alternative embodiments, the respiratory gas can also be used directly from the respiratory gas outlet 4 for the respiration of the patient, which can be in the form of a hose port for this purpose.

The connection to the patient can be realized via one or more respiration hoses (not illustrated here) and/or a suitable patient interface. The respiration hoses and/or the patient interface can usually be connected to the hose port 53 of the ventilator 50. In some embodiments, the respiration hoses and/or the patient interface can also be connected directly to the respiratory gas outlet 4 of the respiratory gas humidifier 1. The patient interface may be, for example, a respiration mask, a tracheal tube or a tracheal cannula, with which the warmed-up and/or humidified respiratory gas can be fed from the respiratory gas humidifier 1 to the respiratory system of the patient.

The respiratory gas outlet 4 can be arranged on the lower housing part 23 and/or formed thereby. The respiratory gas outlet 4 can—when the respiratory gas humidifier 1 is standing as intended on the bottom 24 of the lower housing part 23—be arranged below the liquid surface. The respiratory gas outlet 4 is arranged preferably adjacent to the bottom 24. The respiratory gas outlet 4 is arranged low enough, as seen in the vertical direction, that a tensile force that is applied thereto cannot cause the device to wobble or tip, or can cause this only with difficulty. The respiratory gas outlet 4 is arranged low enough, as seen in the vertical direction, that the hose port 53 of the ventilator can also be arranged low down. Thus, the line 54 in the device can be configured to be very short, and in particular have no vertical deflection.

In order to humidify the respiratory gas, the respiratory gas is guided along the respiratory gas path 2 through a water chamber 5. The water chamber 5 is configured to confer humidity and/or a temperature on the respiratory gas. The lower housing part 23 may be in the form of a water chamber 5 or comprise the water chamber 5. The water chamber 5 is a container for storing a liquid 18 with which the respiratory gas can be humidified. The liquid 18 may be, for example, water or an aqueous liquid. The water chamber 5 may have an electric heating element 17 for warming up the liquid 18 from the water chamber 5. The respiratory gas humidifier 1 and/or the ventilator 50 may comprise a control device (not illustrated here) with which the electric heating element 17 can be controlled in order to set a particular temperature. The heating element 17 may be in the form, for example, of an electric heating rod which can be introduced into the liquid 18.

The water chamber 5 may be configured to conduct the respiratory gas flow as close as possible over the liquid surface. For this purpose, it is possible for at least one flow conducting element 15 to be arranged in the water chamber, said flow conducting element being designed to conduct the respiratory gas in the direction of the liquid surface. The respiratory gas humidifier 1 is configured such that the respiratory gas flows along the respiratory gas path 3 from the respiratory gas inlet 2 to the respiratory gas outlet 4. In the process, the respiratory gas comes into contact with the liquid 18 in the evaporated and/or heated state, with the result that the respiratory gas is humidified and/or brought to temperature.

In order to create a respiratory gas flow in the respiratory gas humidifier 1, the respiratory gas humidifier 1 may comprise at least one respiratory gas drive (not illustrated here), for example a fan. The respiratory gas drive may be arranged in or on the housing of the respiratory gas humidifier 1. Thus, the respiratory gas humidifier 1 itself may be in the form of a ventilator 50. It is preferred, however, for the respiratory gas flow, alternatively or additionally, to be created by a respiratory gas drive of the ventilator 50.

In order to control the operation of the respiratory gas humidifier 1, in particular the heating element 17, the respiratory gas humidifier 1 may comprise a control device (not illustrated here). The control device may be arranged in or on the housing of the respiratory gas humidifier 1. It also possible, however, for the respiratory gas humidifier 1, alternatively or additionally, to be controlled by a control device of the ventilator 50.

The water chamber 5 may have a water chamber inlet 6 and a water chamber outlet 7. The respiratory gas can flow into and back out of the water chamber 5 through these. Formed between the water chamber outlet 7 and the respiratory gas outlet 4 is a channel 8 for conducting the humidified and/or warmed-up respiratory gas to the respiratory gas outlet 4. The channel 8 may be formed at least partially by the upper housing part 21 and/or the lower housing part 23 or extend through same.

The respiratory gas humidifier 1 may—as shown in this example—also have a central part 22 (illustrated in a chequered manner in FIG. 1). The central part 22 may be arranged in a respiratory-gastight manner between the upper housing part 21 and the lower housing part 23. The channel 8 may be formed at least partially by the central part 22 or extend through same. The channel 8 may be formed at least partially (only) by a connection of the upper housing part 21 to the lower housing part 23 or by a connection of the upper housing part 21 to the lower housing part 23 and to the central part 22.

The upper housing part 21, lower housing part 23 and/or central part 22 may be connected reversibly together. The housing parts 21, 22, 23 may thus, for example for cleaning, be separated from one another and subsequently connected together again. As a result of the separation of the housing parts 21, 22, 23, the channel 8 can also preferably be at least locally opened, in order for it to be possible to clean it more easily. In some embodiments, the channel 8 may comprise a(n additional) heating device, which is configured to heat the channel 8 to a first temperature in order to avoid condensation and/or to a second temperature in order to kill off and/or inactivate germs (this not being shown).

The channel 8 may, as seen in its longitudinal direction, be formed at least partially in a straight manner and/or at least partially in an angled and/or bent manner. As shown in this example, the channel 8 may, as seen in its longitudinal direction, comprise one or more channel portions 9, 10, 11, 12 that are fluidically connected together.

A first channel portion 9 may be configured to be connected to the water chamber outlet 7. A second channel portion 10 may be configured to be connected to the respiratory gas outlet 4. Furthermore, one or more further channel portions 11, 12 may be arranged between the first channel portion 9 and the second channel portion 10 and connect these together. The channel portions 9, 10, 11, 12 are connected pneumatically together and are configured to effect at least one deflection of the respiratory gas in the channel 8.

In this example, the respective deflection angle may be +/−90° such that vertical and horizontal channel portions alternate. In this case, the first channel portion 9 may be arranged at least largely vertically. As a result, the humidified and/or warmed-up respiratory gas can first of all be conducted away from the liquid surface in a vertical direction. The following third channel portion 11 may be arranged at an angle of 90° to the first channel portion 9, such that it is arranged at least largely horizontally.

The fourth channel portion 12 following the third channel portion 11 may in turn be arranged at an angle of 90° to the previous channel portion 11, such that it is arranged at least largely vertically. As a result, the channel 8 can be designed to conduct the respiratory gas vertically in the direction of the bottom 24. The second channel portion 10 following the fourth channel portion 12 can effect a further deflection of the respiratory gas into the horizontal direction. Thus, the respiratory gas can be released from the respiratory gas humidifier 1 via the second channel portion 10 and the respiratory gas outlet 4.

In this example, the second channel portion 10, which leads to the respiratory gas outlet 4, is arranged lower down, as seen in the vertical direction, than the remaining channel portions 9, 11, 12 and the water chamber outlet 7. The channel 8 is thus configured for arranging the respiratory gas outlet 4 as low down as possible in the respiratory gas humidifier 1. The third channel portion 11 may, as seen in the vertical direction, represent the highest-up portion of the channel.

In a simple exemplary embodiment, the water chamber inlet 6 may be arranged in the upper housing part 21. Then, the water chamber inlet 6 may be the respiratory gas inlet 2 (this not being shown). It is also possible, as shown, for a chamber 14 to be formed between the water chamber inlet 6 and the respiratory gas inlet 2. Then, the respiratory gas path 3 can lead from the respiratory gas inlet 2 into the chamber 14 and from the latter to the water chamber inlet 6 and to the water chamber 5.

The water chamber inlet 6 may be in the form of an opening between the upper housing part 21 and the lower housing part 23 (this not being shown). The water chamber inlet 6 may, as shown in this example, also be in the form of an opening in the central part 22 or between the central part 22 and the lower housing part 23. The water chamber inlet 6 may also be in the form of a water chamber inlet channel 16 with its own volume.

In this example, a cross section (orthogonal to the direction of flow) of the chamber 14 is larger than a cross section of the respiratory gas inlet 2 and/or a cross section of the water chamber inlet 6 or of the water chamber inlet channel 16 and/or a cross section of the channel 8. A cross section of the water chamber 5 is also, in this example, larger than the cross section of the respiratory gas inlet 2 and/or of the water chamber inlet 6 or of the water chamber inlet channel 16 and/or of the channel 8. Thus, the immediately adjoining portions of the respiratory gas path 3 differ with regard to the size of their cross section orthogonal to the direction of flow. Preferably, the cross section and/or the shape of the respiratory gas path 3 abruptly increases or decreases in size at a transition between the adjoining portions. The chamber 14 and/or the channel 8 and/or the water chamber 5 and/or the water chamber inlet 6 or the water chamber inlet channel 16 may have different dimensions such that their respective acoustic impedances differ from one another. This can result in a reduction in the operating noise of the respiratory gas humidifier 1.

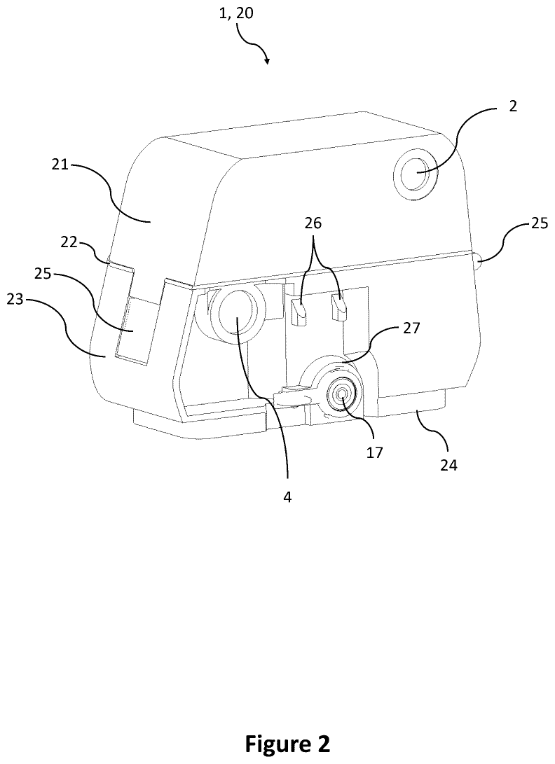

FIG. 2 shows a perspective exterior view of a respiratory gas humidifier according to one embodiment of the invention. It is apparent from FIG. 2 that the respiratory gas outlet 4 is arranged on the lower housing part 23. In this example, the respiratory gas inlet 2 is arranged on the upper housing part 21. In this example, the respiratory gas outlet 4 is arranged closer to the bottom 24, as seen in a vertical direction, than the respiratory gas inlet 2. The heating element 17 is likewise arranged on the lower housing part 23. The lower housing part 23 may have a heating element receptacle 27, via which or by way of which the heating element 17 can be received on the lower housing part 23. The heating element 17 may be connected reversibly to the lower housing part 23 via a mechanical closure. For example, the heating element 17 may be introduced into or fastened in the lower housing part via a rotary closure, preferably via a bayonet closure. In the exemplary embodiment shown, the respiratory gas inlet 2 and the respiratory gas outlet 4 are located on the same side face of the respiratory gas humidifier 1. It is also possible for the heating element 17 to be arranged on the same side face.

The upper housing part 21, lower housing part 23 and/or central part 22 may be connected reversibly together. The connection is preferably entirely respiratory-gastight. For this

purpose, the respiratory gas humidifier 1 may have (elastomeric) sealing faces (not illustrated here) which are arranged on the contact faces of the different housing parts 21, 22, 23. The upper housing part 21, lower housing part 23 and/or central part 22 may be manufactured from or comprise an elastomeric material.

In addition, the respiratory gas humidifier 1 may have at least one locking device 25. For assembly, the upper housing part 21 can be placed on the lower housing part 23 and optionally locked. In an embodiment with a central part 22, this can be clamped in place between the upper housing part 21 and lower housing part 23. For assembly, the central part 22 can be inserted into the lower housing part 23. Then, the upper housing part 21 can be placed on the central part 22 and/or the lower housing part 23 and locked.

At the connecting points between the upper housing part 21, lower housing part 23 and/or central part 22, elastomeric sealing faces (not illustrated here), made for example of silicone, may make the connection respiratory-gastight.

The locking device 25 may lock the upper housing part 21 and lower housing part 23 together. The locking device 25 may be formed, for example, as a latching mechanism with engagement hooks on the one housing part and corresponding openings in the other housing part. To release the connection, a gripping surface may be provided, which, upon a slight pulling movement, releases the latching connection on account of a certain elasticity of the housing materials that are used.

The respiratory gas humidifier 1 may be connected to a ventilator 50 (not illustrated here) via the respiratory gas inlet 2 and/or the respiratory gas outlet 4. For this purpose, one or more fastening devices (not shown here) may be arranged on the housing of the respiratory gas humidifier 1 and/or on the ventilator 50. The fastening may be fixed mechanically and/or magnetically and/or electrically. The fastening is formed preferably reversibly, such that easy fastening to and releasing from the ventilator 50 can be performed. The fastening devices may be in the form, for example, of hooks on the ventilator 50 with corresponding counterparts on the respiratory gas humidifier 1, or vice versa. The hooks may be designed to be hooked or clipped, or the like, in the corresponding counterpart. The respiratory gas humidifier 1 may furthermore comprise at least one fastening ramp 26. The fastening ramps 26 may serve as ramps for pulling the respiratory gas humidifier 1 off the ventilator 50. At least one opposing ramp may be arranged on the ventilator 50, said opposing ramp being designed such that, upon a vertical movement of the unlocking mechanism, it results in a horizontal movement (detachment) of the respiratory gas humidifier 1.

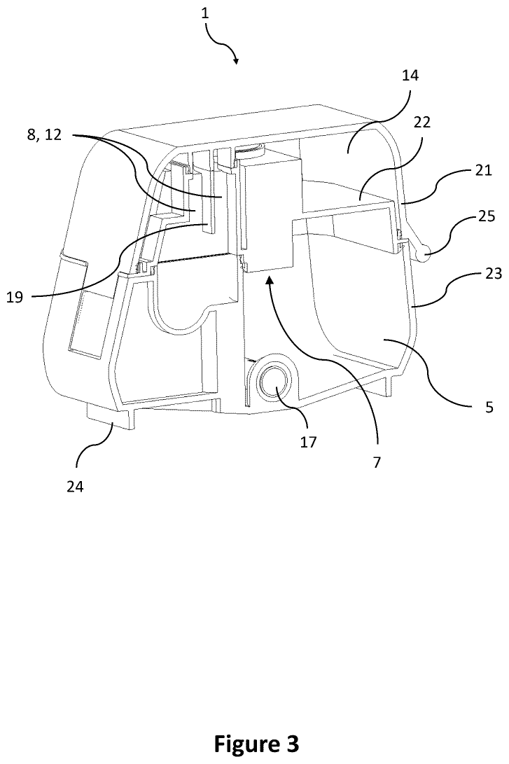

FIG. 3 shows a perspective cross section of a respiratory gas humidifier according to one embodiment of the invention. This example shows that the central part 22 can be clamped in place between the upper housing part 21 and the lower housing part 23. The central part 22 is clamped in place in a respiratory-gastight manner between the upper housing part 21 and the lower housing part 23 such that the respiratory gas can be conducted along the respiratory gas path. The respiratory gas path is formed, in this example, by the upper housing part 21, the lower housing part 23 and the central part 22.

In this exemplary embodiment, the channel 8 at least partially has a separating element 19. The separating element 19 is configured to subdivide the channel 8, as seen in its longitudinal direction, at least partially into at least two sub-channels. As is apparent from this depiction, the fourth channel portion 12 can be subdivided into two sub-channels. The separating element 19 may be configured to promote a laminar flow and to prevent turbulence in the channel 8.

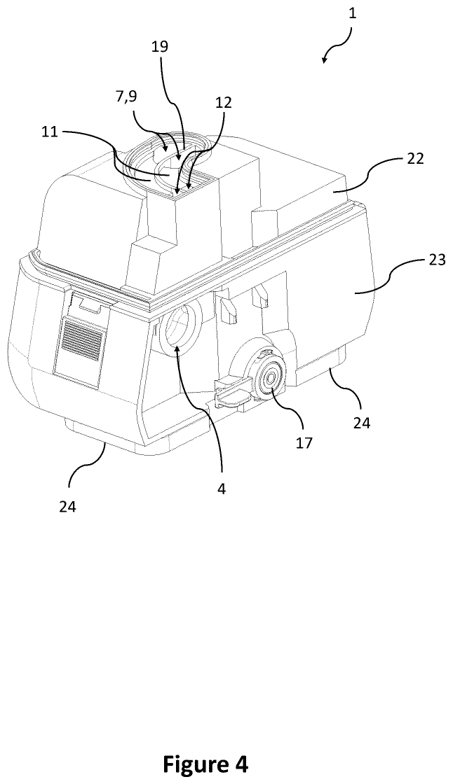

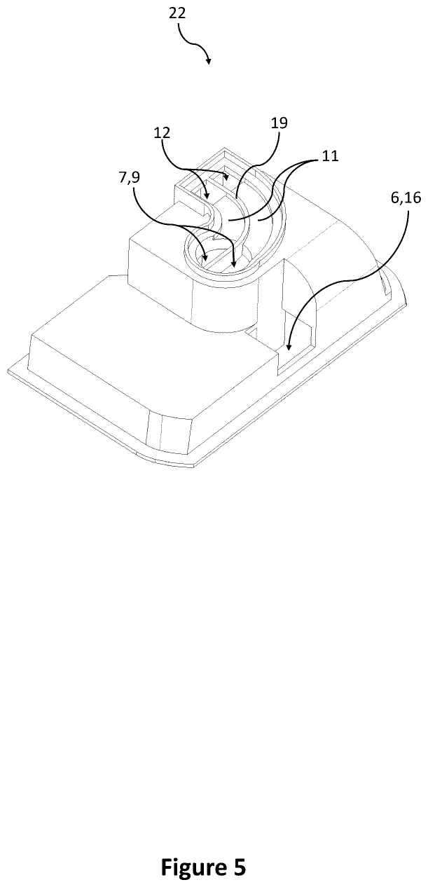

FIG. 4 shows a perspective exterior view of a respiratory gas humidifier without the upper housing part being illustrated, according to one embodiment of the invention. FIG. 5 shows a perspective exterior view of a central part of a respiratory gas humidifier according to one embodiment of the invention.

In this example, the water chamber outlet 7 and the first channel portion 9 are arranged centrally in the device. It is apparent from FIGS. 4 and 5 that the separating element 19 may, in some embodiments, subdivide a plurality of sub-portions of the channel 8 or the entire channel 8 into two sub-channels. In this example, the separating element 19 subdivides at least the first channel portion 9, the third channel portion 11 and the fourth channel portion 12 into two sub-channels. Alternatively or additionally, the separating element 19 can also subdivide the water chamber outlet 7 and/or the second channel portion 10 into two portions or sub-channels. The arrangement of a plurality of separating element 19 to create further sub-channels is also possible (this not being shown).

In the exemplary embodiment according to FIGS. 3 to 5, it is furthermore shown that the channel 8, as seen in its transverse direction, can be formed at least locally in a bent manner. It is apparent that, for example, the third channel portion 11, as seen in its transverse direction, is formed in a bent manner. As a result, a three-dimensional configuration of the channel 8 and thus a compact design is realized.

It is apparent from FIG. 4 and FIG. 5 that the channel 8 is formed as a whole only by a connection of the housing parts. The channel 8 may, for example, be formed at least locally in two parts, such that the channel can be opened at least locally when the housing parts are separated. This makes it easier to clean the channel 8. The channel 8 may be formed at least partially only by a connection of the upper housing part 21 to the lower housing part 23 or by a connection of the upper housing part 21 to the lower housing part 23 and to the central part 22.

The figures show that locally half of the channel is formed by the central part 22, in this case the third channel portion 11. The second half of the channel 8 can be formed by the upper housing part 23 (not shown here). Thus, the channel 8 is formed fully and/or in a respiratory gas-conducting manner only when the housing parts are connected together.

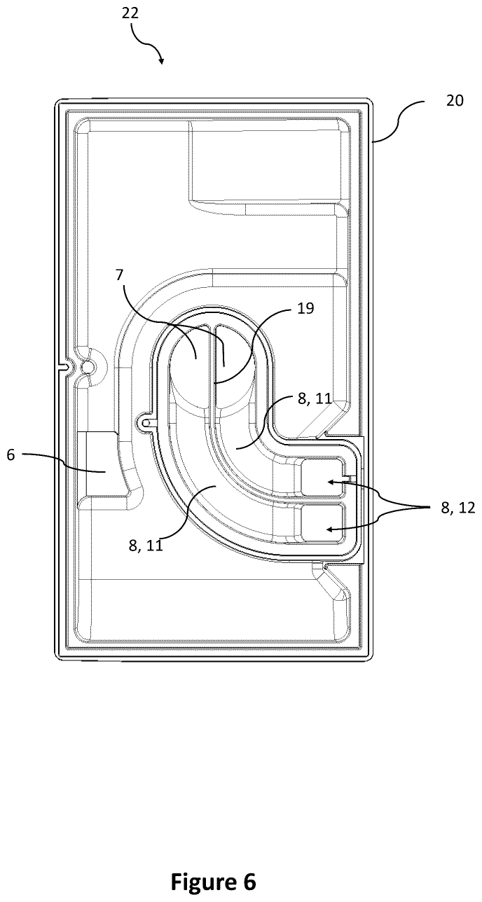

FIG. 6 shows a view of a central part 22 from above according to one embodiment of the invention. It is apparent from FIG. 6 that the central part 22 can at least partially form the water chamber inlet 6 and the water chamber outlet 7. Moreover, the central part 22 can at least partially form the channel 8 at least locally, wherein, in this depiction, the third channel portion 11 can be seen.

The water chamber inlet 6 may be in the form of a (simple) opening in the central part 22. The water chamber inlet 6 may also comprise a volume and be in the form of a channel, and thus be in the form of a water chamber inlet channel 16 in the central part 22. The respiratory gas can pass along the respiratory gas path 3 into the water chamber 5 through the water chamber inlet 6 or the water chamber inlet channel 16. The water chamber inlet 6 or the water chamber inlet channel 16 can, as shown, be arranged adjacent to the outer wall of the housing 20.

The water chamber outlet 7 may likewise be in the form of a (simple) opening in the central part 22. The channel 8 may lead, at its one end, into the water chamber outlet 7. The respiratory gas can pass along the respiratory gas path 3 out of the water chamber 5 and into

the channel 8 through the water chamber outlet 7. The water chamber outlet 7 or the channel 8 may be configured to conduct the warmed-up and/or humidified respiratory gas out of the water chamber 5 to the respiratory gas outlet 4 (not illustrated here).

The water chamber outlet 7 may, as shown, be arranged centrally. Then, the water chamber outlet 7 can be arranged in a manner spaced apart as far as possible from the outer walls of the housing 20. This affords the advantage that the liquid from the water chamber 5 cannot pass into the channel 8, or can do so only with difficulty, when the respiratory gas humidifier passes into an inclined position. The channel 8 may be configured to conduct the respiratory gas outwardly from the middle, i.e. in the direction of the housing wall 20. The channel 8 leads at its other end into the respiratory gas outlet 4.

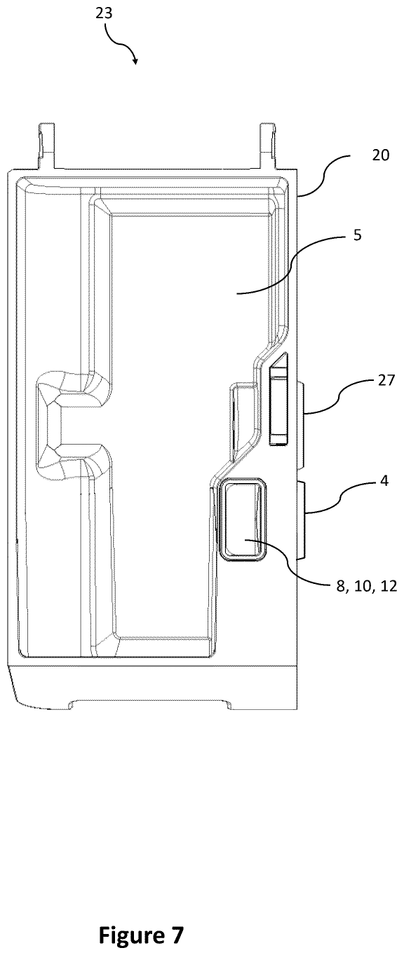

FIG. 7 shows a view of a lower housing part 23 from above according to one embodiment of the invention. It is apparent from FIG. 7 that the lower housing part 23 can comprise the water chamber 5. In addition, the lower housing part 23 can at least partially form the channel 8 at least locally. It is apparent from this figure that the lower housing part 23 can comprise at least one sub-portion of the channel 8. The lower housing part 23 may preferably comprise or form those channel portions 10, 12 that are arranged adjacent to the respiratory gas outlet 4 or lead into the latter.

The channel 8 has a channel wall and is formed via the latter so as to be separated from the water chamber 5 in a watertight manner. The channel 8 is configured to conduct the warmed-up and/or humidified respiratory gas out of the water chamber 5 in the direction of the

respiratory gas outlet 4. It is apparent from FIG. 7 that the respiratory gas outlet 4 is arranged on the lower housing part 23 or can be formed thereby. The respiratory gas outlet 4 may be arranged adjacent to the heating element receptacle 27. Through the heating element receptacle 27, a heating element (not shown here) can be introduced into or mounted in the water chamber 5. The respiratory gas outlet 4 and heating element receptacle 27 may preferably be arranged on the same housing wall.

Finally, it should be noted that terms such as “have”, “comprise”, “include”, “having” etc. do not exclude other elements or steps, and indefinite articles such as “a” or “an” do not exclude a plurality.

Furthermore, it should be noted that features or steps that are described with reference to one of the embodiments above can also be used in combination with features or steps that are described with reference to other embodiments from among the embodiments above.

### LIST OF REFERENCE NUMERALS

1 Respiratory gas humidifier
2 Respiratory gas inlet
3 Respiratory gas path
4 Respiratory gas outlet
5 Water chamber
6 Water chamber inlet
7 Water chamber outlet
8 Channel
9 First channel portion
10 Second channel portion
11 Third channel portion
12 Fourth channel portion
14 Chamber
15 Flow conducting element
16 Water chamber inlet channel
17 Heating element
18 Liquid
19 Separating element
20 Housing
21 Upper housing part
22 Central part
23 Lower housing part
24 Bottom
25 Locking device
26 Fastening ramp
27 Heating element receptacle
50 Ventilator
51 First port
52 Second port
53 Hose port
54 Line

What is claimed is:

1. A respiratory gas humidifier, wherein the humidifier comprises a housing comprising an upper housing part and a lower housing part with a water chamber for humidifying and/or warming up a respiratory gas and further comprises a respiratory gas inlet and a respiratory gas outlet, wherein a respiratory gas path for conducting the respiratory gas from the respiratory gas inlet to the respiratory gas outlet is formed between the respiratory gas inlet and the respiratory gas outlet and is guided through the water chamber and comprises a watertight channel for conducting the humidified and/or warmed-up respiratory gas, said channel leading into the water chamber at one end and into the respiratory gas outlet at another end and being configured such that the respiratory gas outlet is arranged on the lower housing part and/or is formed at least partially thereby.

2. The respiratory gas humidifier of claim 1, wherein the respiratory gas outlet—when the water chamber has been filled as intended with a liquid for humidifying the respiratory gas—is arranged below the liquid surface.

3. The respiratory gas humidifier of claim 1, wherein the channel is formed at least partially by the upper housing part and/or the lower housing part or extends through same.

4. The respiratory gas humidifier of claim 1, wherein the respiratory gas humidifier comprises a central part which is arranged in a respiratory-gastight manner between the upper housing part and the lower housing part, the channel being formed at least partially by the central part or extending through same.

5. The respiratory gas humidifier of claim 1, wherein the channel is formed at least partially by a connection of the upper housing part to the lower housing part or by a connection of the upper housing part to the lower housing part and to a central part arranged in a respiratory-gastight manner between the upper housing part and the lower housing part.

6. The respiratory gas humidifier of claim 1, wherein the channel, as seen in its longitudinal direction, is formed at least partially in an angled and/or bent manner.

7. The respiratory gas humidifier of claim 1, wherein the channel, as seen in its longitudinal direction, comprises one or more channel portions that are fluidically connected together, the channel comprising a first channel portion for connecting to a water chamber outlet, a second channel portion for connecting to the respiratory gas outlet, and at least one further channel portion connecting the first channel portion to the second channel portion, the channel portions being configured to effect at least one deflection of the respiratory gas.

8. The respiratory gas humidifier of claim 7, wherein the first channel portion is configured such that—when the respiratory gas humidifier is standing as intended on a bottom and the water chamber has been filled with a liquid for humidifying the respiratory gas—it leads away from a liquid surface in a vertical direction.

9. The respiratory gas humidifier of claim 7, wherein a third channel portion connecting the first channel portion to the second channel portion is configured such that—when the respiratory gas humidifier is standing as intended on a bottom—it is arranged above the respiratory gas outlet as seen in a vertical direction.

10. The respiratory gas humidifier of claim 1, wherein the channel at least partially comprises a separating element which is configured to subdivide the channel, as seen in its longitudinal direction, at least partially into at least two sub-channels.

11. The respiratory gas humidifier of claim 1, wherein the water chamber has a water chamber inlet and a water chamber outlet and the respiratory gas path is guided into the water chamber through the water chamber inlet and out of the water chamber through the water chamber outlet, the water chamber inlet being arranged in the upper housing part and/or in a central part and being in the form of a simple opening or of a water chamber inlet channel.

12. The respiratory gas humidifier of claim 11, wherein the water chamber outlet and/or a first channel portion is/are arranged, as seen in a horizontal plane, in a region of the middle of the respiratory gas humidifier.

13. The respiratory gas humidifier of claim 1, wherein the channel, as seen in its transverse direction, is formed at least partially in an angled and/or bent manner such that the respiratory gas path leads out of a middle of the respiratory gas humidifier as far as the housing and/or as far as the respiratory gas outlet.

14. The respiratory gas humidifier of claim 1, wherein the respiratory gas humidifier further comprises a chamber which is formed between the respiratory gas inlet and a water chamber inlet and is part of the respiratory gas path, a cross section of the chamber being larger than a cross section of the respiratory gas inlet and/or a cross section of the water chamber inlet or of the water chamber inlet channel and/or of the channel.

15. The respiratory gas humidifier of claim 14, wherein the chamber formed between the respiratory gas inlet and a water chamber inlet and/or the channel and/or the water chamber and/or the water chamber inlet or the water chamber inlet channel have acoustic impedances and have different dimensions such that their respective acoustic impedances differ from one another.

16. A ventilator, wherein the ventilator comprises a respiratory gas duct, a first port and a second port, and the respiratory gas humidifier of claim 1, and wherein the respiratory gas humidifier is connected to the respiratory gas duct of the ventilator via a respiratory gas inlet at the first port and via a respiratory gas outlet at the second port.
